(19) United States
(12) Patent Application Publication
(10) Pub. No.: US 2003/0149358 A1
(43) Pub. Date: Aug. 7, 2003

(54) Ultrasonic system for non-invasive early prostate cancer detection

(76) Inventors: Nissan Maskil, Herzelia (IL); Eran Eilat, Herzelia (IL)

Correspondence Address: DR. D. GRAESER LTD., C/O THE POLKINGHORNS, 9003 FLORIN WAY, UPPER MARLBORO, MD, 20772, US

(21) Appl. No.: 10/066,754

(22) Filed: Feb. 6, 2002

(51) Int. Cl. A61B 8/00

(52) U.S. Cl. 600/437

(57) ABSTRACT

An ultrasonic hand held device for non-invasive diagnosis of early prostate cancer. The device analyzes the consistency of tissue density and also measures blood velocity characteristics in the prostate gland. The device is preferably pen-shaped for ease of insertion to the rectum, and also is preferably operable by the patient, such that skilled medical personnel and/or a specialized medical setting are not required for operation.

### FIELD OF THE INVENTION

The present invention is of a device and method for the detection of prostate cancer in a subject with a non-invasive, easy to operate device.

### BACKGROUND OF THE INVENTION

In the United States, prostate cancer is the most common solid tumor malignancy in men. Prostate cancer is second leading cause of cancer deaths in American men. In 1997 approximately 210,000 new cases of prostate cancer were diagnosed and more than 41,800 deaths were attributed to this malignancy. At present, chemotherapy and immunotherapy cannot adequately treat prostate cancer once it has spread beyond the prostate gland. Therefore, curative treatment for localized tumors may be the best hope of lowering mortality rate for prostate cancer patients. Thus, the most rational solution for the situation is the early diagnosis of this malignancy, which could be a “life saving” procedure.

The diagnosis of prostate cancer is determined in a biopsy from the tissue which shows the typical malignant cells. However, biopsy is a complex method that involves an expert urologist, and has complications. Currently, there are a few diagnostic aids to assist the decision for performing a biopsy. One such aid is the digital rectal examination, which could detect up to 50% of the prostate cancer cases and is recommended by the American Cancer Society; however this examination is uncomfortable for the patient. Many men are reluctant to undergo this annual examination and therefore are not properly screened.

Prostate specific antigen (PSA) is a blood test, which has a sensitivity of about 67.5% to 80% in patients with normal serum PSA levels (defined as 4.0 ng/mL or less). However, the PSA test is not very specific, since only one third of men with an abnormal serum PSA level actually have cancer. Once initial prostate cancer screening has been performed, the physician must decide how often the patient should be reevaluated. Compared with one-time screening, yearly digital rectal examinations and PSA testing detect a higher proportion of organ-confined cancers. Almost 75 percent of patients with prostate cancer detected on serial screenings are found to have organ-confined and therefore potentially curable cancer, compared with 66 percent of patients who undergo only a single screening. The American Cancer Society recommends that, beginning at the age of 50 years, men with a life expectancy of at least 10 years should be offered annual digital rectal examinations and PSA tests. Alternatively, some investigators have suggested that examinations may need to be performed only every other year in patients with a serum PSA level of less than 2 ng per mL, given the extremely low incidence of cancer found in this group.

The central concept that tumor growth is “angiogenesis dependent” is well accepted today, with more than 2,500 reports showing angiogenesis linked to tumor growth. It is also well acceptable that “every increment of tumor growth requires an increment of vascular growth” (references 4, 5, 6, 7).

Prostate cancer is a relatively “slow growing” tumor; its doubling time is 2-6 years. Generally, shorter doubling times indicate a more aggressive tumor (reference 8). Therefore, it would be advantageous to be able to monitor blood flow changes over a duration of several years in order to be able to assess the development of prostate cancer.

Trans rectal ultrasound was thought to have value in screening for prostate cancer. However, experimentation has shown that this type of technology has a relatively poor ability to detect disease. The trans rectal Doppler examination is a newer technology which compares the blood flow velocities in the prostate gland. Cancer tissue shows increased blood flow at a very early stage, since blood is needed for the tumor to grow.

The Radiology Society of North America stated that power Doppler exams of the prostate were the most effective method of detecting early prostate carcinomas. Areas of increased flow are 4.7 times more likely to contain cancer. The negative predictive value is 99%, so this exam is being used to prevent unnecessary biopsies. However, currently this type of examination can only be performed at a hospital or clinic, as it requires highly skilled personnel to operate the necessary equipment in order to perform the examination.

Any type of procedure which permits multiple, periodic examinations to be performed would be useful, since prostate cancer has a relatively slow doubling time (approximately three years). Therefore, regular annual examinations would be highly useful, but are typically not performed, even for men in higher risk groups.

### SUMMARY OF THE INVENTION

The background art does not teach or suggest a device, which can be used by the patient or subject, without expert medical intervention. In addition, the background art does not teach or suggest a device which includes the measurement of Doppler at multiple frequencies and co-registration of both Ultrasound Doppler and density measurements.

The present invention is of an ultrasound hand held device for non-invasive diagnosis of early prostate cancer. The device analyzes the consistency of tissue density and measures blood flow velocity characteristics in the prostate gland. The device preferably comprises a pen-shaped portion, and more preferably features the pen-shaped portion connected to a base for ease of insertion to the rectum, and also is preferably operable by the patient, such that skilled medical personnel and/or a specialized medical setting are not required for operation. Connecting the pen-shaped portion to the base enables subsequent measurements to be made from the same angle, which improves the performance of the device, particularly with regard to the detection of change(s) in the tissue.

Doppler detection is performed by receiving a plurality of signals. The frequency shift in the reflected signal induced by moving (blood) particles is presented in the images in different frequencies, increasing the amount of transmitted power and the signal to noise ratio (SNR). One ensemble of received signals is processed in the time and the frequency domain to estimate motion at each point in space along an ultrasound line. This process is repeated over some ultrasound frequencies, in order to obtain the average measured parameters in the space domain that cover the main blood vessels in the prostate.

The received signal is sampled by 16-bit Analog to Digital converter (ADC). The sampled data is received for Doppler analysis, which computes FFT (Fast Fourier Transform) of the data in order to process the data in the time and frequency domains. The demand for adequate frame rates, diagnostic resolution, dictate that an ultrasound system typically transmits from 2 to about 64 pulses to detect any distinct point in space. This constraint may create estimate dropouts in flow fields and demand extensive spatial and/or temporal averaging of estimates, which may result in inaccurate differentiation between flowing blood and tissue. This is optionally and preferably overcome by co-registration of both Power Doppler and Ultrasound density measurements, and allocating both results on the same prostate grid. The co-registration results are preferably compared in a plurality of consecutive measurements in order to identify characteristic features that can optionally and more preferably be monitored in order to detect changes.

For example, U.S. Pat. No. 4,534,357 discloses a system that transmits broad, single band, pulses and then processes receive signals from two narrow receive bands for each broadband pulse transmitted. In other examples, U.S. Pat. Nos. 5,046,500 and 5,183,047 both describe systems that transmit pulses composed of two separate narrow frequency bands, and then process signals from corresponding two narrow receive bands for each transmitted pulse.

There are several forms of depiction of blood flow in medical Doppler imaging: color Doppler, pulsed Doppler, and power Doppler. Color Doppler provides an estimate of the mean velocity of flow within a vessel by color coding the information and displaying it superimposed on the gray-scale image. The flow direction is arbitrarily assigned the color red or blue, indicating flow toward or away from the transducer, respectively. Pulsed Doppler allows a sampling volume (or gate) to be positioned in a vessel visualized on the gray-scale image, and displays a spectrum, or graph, of the full range (as opposed to the mean velocity, as in color Doppler ultrasound) of blood velocities within the gate plotted as a function of time. The amplitude of the signal is approximately proportional to the number of red blood cells and is indicated as a shade of gray. Color Doppler provides a global depiction of blood flow in a region and may be used as a guide for the subsequent placement of the pulsed Doppler gate for detailed analysis at a site of potential flow abnormality.

Power Doppler, which is not routinely used in arterial Doppler evaluation of the lower extremity, depicts the amplitude, or power, of Doppler signals as function of the Doppler shifts. This allows detection of a larger dynamic range of Doppler shifts and thus better visualization of small vessels, but at the expense of directional and velocity information.

With regard to ultrasound imaging of the prostate, it should be noted that diagnostic ultrasound is the most common imaging technology in the urology clinic to investigate patients with problems of the lower urinary tract. It is a cheap and versatile tool to display a cross-sectional overview of the prostate and bladder, and can be used easily to guide the performance of biopsies of suspicious areas. Furthermore, its value to obtain the size of the prostate and to guide the biopsies is unquestioned. The value of transrectal ultrasound for differential diagnosis, however, is not clear, because of the variability in the appearance of malignant tissue in ultrasound images of the prostate.

Sensitivity of traditional gray scale ultrasound for prostate cancer detection is limited. The sensitivity of ultrasound in detecting malignancy in the prostate is not optimal, leading to a large number of unnecessary biopsies. Improvement of the diagnostic accuracy of ultrasound would mean a great step forward in the clinical application of transrectal ultrasound of the prostate.

### BRIEF DESCRIPTION OF THE DRAWINGS

The invention is herein described, by way of example only, with reference to the accompanying drawings, wherein:

FIG. 1 is a schematic block diagram of an illustrative system according to the present invention; and

FIG. 2 shows an exemplary embodiment of the present invention with a pen construction of the device as inserted into the rectum of the subject.

### DESCRIPTION OF THE PREFERRED EMBODIMENTS

The present invention is of an ultrasonic hand held device for non-invasive diagnosis of early prostate cancer. The device analyzes the consistency of tissue density and also measures blood velocity characteristics in the prostate gland and detects changes in the blood flow. The device preferably comprises a pen-shaped portion, and more preferably features the pen-shaped portion connected to a base for ease of insertion to the rectum, and also is preferably operable by the patient, such that skilled medical personnel and/or a specialized medical setting are not required for operation. Connecting the pen-shaped portion to the base enables subsequent measurements to be made from the same angle, which improves the performance of the device, particularly with regard to the detection of change(s) in the tissue. The pen-shaped portion and the base are optionally constructed of plastic. The pen-shaped portion is more preferably constructed of flexible plastic.

It should be noted that “pen-shaped” is not intended to be limiting in any way, as the portion to be inserted into the rectum should be of suitable dimensions for insertion thereof. Also, preferably the pen-shaped portion is connected to the base at a fixed angle, which may optionally be adjusted according to the individual user.

According to preferred embodiments of the present invention, the device uses the ultrasound technique to evaluate at least one of, and more preferably both, the consistency of tissue and the blood velocity at the direction of the tip of the portion to be inserted. The insertion portion of the device preferably has directional antenna mounted to transmit complex ultrasound signals. The return signal is then preferably analyzed by measuring the intensity, phase, and frequency of the backscattered Ultrasound signal. Optionally and more preferably, the procedure is performed more than once, in order to be able to compare the current results to previous data by detecting changes, which increases the probability of detecting tissue changes in the region of interest (ROI) in the prostate. Furthermore, the preferred embodiment relates to serial measurements and the comparison of the most recently performed measurement to the previous one, such a comparison could detect changes at an earlier stage. Examinations preformed by urologists and rentgenologists do not take the above into consideration, hence are less sensitive to the cancerous change in the prostate.

The combination of measuring tissue density with comparison of blood flow could significantly increase sensitivity and specificity of the invention.

More preferably, in order for the device to be inserted correctly, the portion of the device to be inserted is mounted on a plastic seat. Therefore, when the patient sits on the seat, the pen (insertion portion of the device) is inserted in the same direction and penetration depth. Also preferably, the results are displayed in a common scale display (no image display) or numeric display for the ease of use by the patient or subject or can be transmitted via telephone to the diagnostic center.

According to other preferred embodiments of the present invention, the device uses trans Doppler ultrasound technology, which has been previously shown to have an overall sensitivity of 74%, specificity 96%, and positive predictive value of 74%, when used for prostate cancer with specific calculation of (see for example O. E Franco, K. Arima, M. Yanagawa and J. Kawamura, BJU International (2000), 85, 1049-1052—The usefulness of power Doppler ultrasonography for diagnosing prostate cancer: histological correlation of each biopsy site). The trans Doppler method compares the blood flow velocities in the prostate gland. The technology of Doppler ultrasound itself is used primarily for the hemodynamic assessment of blood vessels, arteries and veins. The Doppler technology is used as Duplex imaging. Doppler provides the quantitative data color flow delivers quick detection of power Doppler.

The Doppler shift is detected as a change in the frequency of the reflected ultrasound wave. It is due to the relative motion between the reflector (blood) and transducer (Krebs et al., 1993). The equation below describes this relationship (where D is the Doppler Frequency, V—the velocity, @—the angle of incidence between the beam and interface, c—the velocity of sound in the medium). Even though ultrasound gives only one-dimensional velocity information, volumetric flow can be accurately extrapolated with no angle corrections (by using the reference power Doppler we are not extrapolating the volume).

$$D = \frac{2 VF cos @}{c}$$

The usefulness of Doppler Ultrasound in diagnosis of blood vessel health is apparent upon analysis of the above equation. The ultrasound waves create laminar flow in the blood vessels with the highest velocity in the center of the vessel and decreasing velocity closer to the blood vessel wall. When a curve or plaque is present in the vessel, the flow pattern is altered. Arteries normally have forward, reverse, and second flow components. Abnormal flow is represented by an increased, decreased, or absent component of second flow and increase or absence of reverse flow, such a change in general and increased flow in particular could be the initial imaging finding in the development of cancerous tissue. The relative intensity can be used to measure the relative velocity (although only the radial velocity is measured). Cancer tissue shows increased blood flow at a very early stage, since blood is needed for the tumor to grow.

The method of the present invention preferably includes transmitting ultrasonic energy into the prostate in consecutive transmit events, wherein different transmit events comprise a first transmit event focused at a greater depth layer and a second transmit event focused on the depth in consecutive time frame. More preferably, the method of the present invention includes the use of more than two measurements to average the results of the blood intensity and velocity.

The present invention more preferably features a time varying filter which passes the component portions of the receive beams and the fundamental component of portions of the receive beams. Digital filters are also preferably operative to selectively pass the components of the received beams.

According to another embodiment of the present invention, there is provided a method for Doppler processing of signals in different frequency bands for improved accuracy. Signals from two or more unique frequency bands are preferably processed separately to detect motion. Such processing is more preferably performed after the performance of FFT in the time and frequency domains. Most preferably, after FFT has been performed on the data, the data is further processed by using Doppler derivatives in the ROI (first, second and third), and/or low and and/or band pass filters to locate the discontinuities and/or separations in the vessels.

Using signals at different frequency bands allows for more effective use of unused bandwidth to increase information content and improve estimation accuracy. The transmitted energy consists of a pattern of frequencies (as LFM (linear frequency modulation) or binary frequencies), which may be within the transmitted fundamental frequency band or alternatively within harmonic frequency bands of the band. The increased information content generated by using separate narrow receiving frequency bands is used to improve many aspects of motion detection. The resulting Doppler values of each parameter, such as velocity, energy, and variance parameters, are averaged or otherwise combined.

The parameter selection process can be used to further improve differentiation between blood flow and clutter. Signals at two different frequency bands are also used to further improve detection of velocities beyond the conventional alias limit.

Energies of one frequency band at higher frequencies are exposed to more significant attenuation than energies of a lower frequency band, contributing to inferior SNR and potentially poor velocity estimates in some cases. Energy estimates are used to identify inferior SNR. A detection algorithm determines velocities beyond the minimum SNR is a 1-bit algorithm that detects consistent but weak Doppler signals. The prostate domain is preferably defined as the region of interrogation.

The principles and operation of the present invention may be better understood with reference to the drawings and the accompanying description.

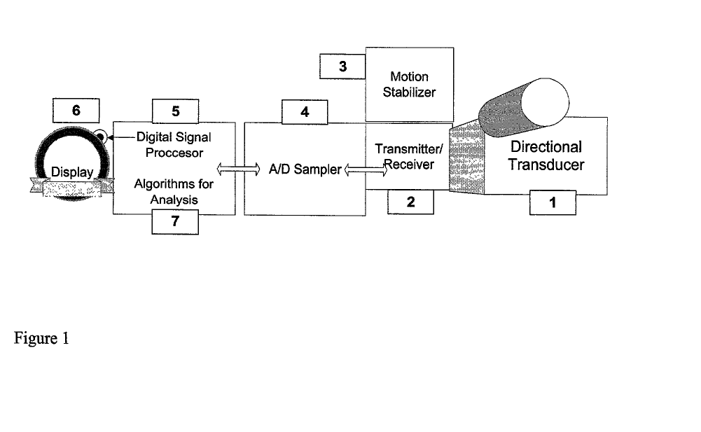

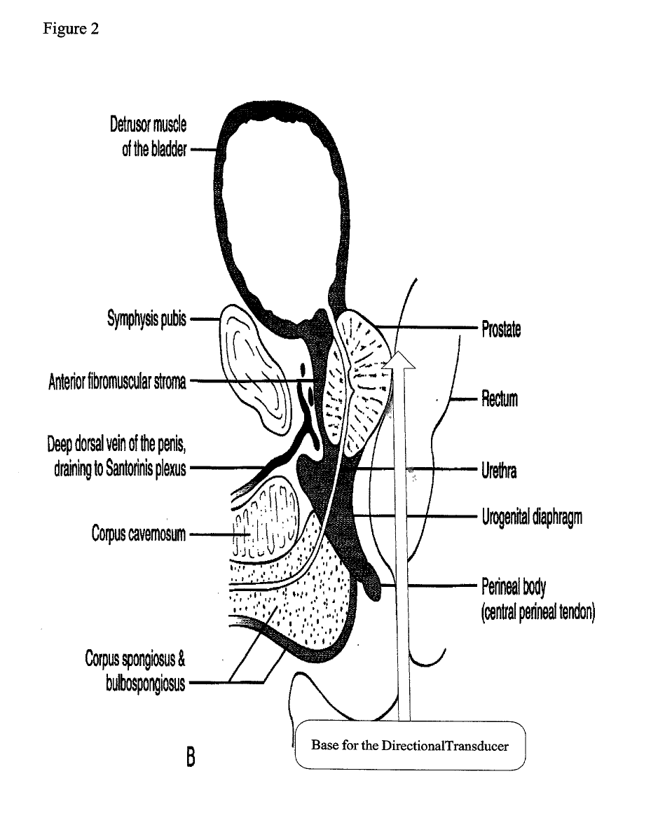

FIG. 1 is a schematic block diagram which shows the main functions of the device of the present invention. A device 10 is preferably a directional ultrasound device that builds the directional beam for transmitting and receiving the signal (FIG. 2 shows device 10 as inserted into the rectum). The beam is preferably built from an array of a minimum of four elements which are mounted on the side of the tip of the device.

A transmitter/receiver 12 is optionally implemented as a plurality of transducers. Transmitter/receiver 12 preferably transmits an ultrasonic signal into the rectum of the subject, in the direction of the prostate, and also detects the resulting back-scattered signal. Transmitter/receiver 12 preferably controls the frequency, phase, shape and intensity of the transmitted pulse. The signal preferably has a (pencil) directional beam shape and frequency modulation with stabilized phase. The basic ultrasound signal is frequency in the bandwidth; this signal is preferably modulated for the present invention in order to change the frequency as a function of time in the pulse duration. More preferably, the signal has frequency with stabilized phase, or a coherency frequency, for coherency integration.

The receiver portion of transmitter/receiver 12 preferably adapts the time, frequency, width and shape of the received pulse. Frequency modulation of the pulse is preferred as it increases the capability to detect the reflected intensity as a function of time and also provides the capability to integrate coherency. The modulation enables measurement, at a short range, of the density of the tissue in improved resolution.

The signal is then preferably passed to an analog to digital (A/D) converter 14 for obtaining digital data for processing, which for example may be up to 16 bits. The digital data is then preferably analyzed with a DSP 16 (Digital Signal Processor).

Transmitter/receiver 12 is optionally implemented with a PW transducer, which both sends and receives the signal (see for example http://www.bae.ncsu.edu/bae/research/blanchard/www/465/textbook/imaging/project s/ultrasound/project/doppler.html). The transducer sends the signal in short bursts, and receives the returned signal when not transmitting. The returned signal is gated so that only information about the desired depth within the body is transmitted. The choice of the sample gate is determined by Nyquist's Theorem. The Doppler shift cannot exceed one half the Pulse Repetition Frequency, or aliasing occurs.

Once the information has been analyzed, the results are optionally displayed on a display 18 for simple numeric or scale presentation.

Device 10 also preferably features a motion stabilizer 20 for determining the direction of the prostate and for detection of changes between sequential tests. Since device 10 is preferably hard mounted on a seat, which may be of plastic for example, device 10 is inserted to a constant position within the rectum of the subject. Motion stabilizer 20 preferably has mechanical sensor that detects small movements, such that measurements are preferably only performed when the sensor is stabilized.

A DSP algorithm 22 could optionally detect the tissue density such as differences between gas, liquid and solids, as well as differences within these groups, such as water and blood, different kind of solids and so forth. In addition, DSP algorithm 22 preferably performs FFT, more preferably with registration of the place of flow with contrast agents to increase delectability in the ROI (region of interest). DSP algorithm 22 may also optionally and more preferably perform cross-correlation between different measurements, with successive measurements more preferably being processed with a different matrix of filters (low pass and high pass).

This data is then more preferably calculated in different RF frequencies per estimation. The change detection algorithm preferably includes the comparison of two-registrant data in the different RF lines, using different derivatives of the Doppler measurement, and optionally and more preferably correlated if possible with the changes in the density measurements. These derivatives of the Doppler results can measure an average weighted blood velocities in the tissue, preferably by measuring two different parameters of the data: density and the power Doppler.

The measurement of density is a conventional ultrasound measurement, which measures the intensity as function of time from the reflected signal. Since a directional beam is obtained from the transducers (transmitter/receiver 12), and the reflected waves from the tissues and substances can be analyzed, density can be determined. Transmitter/receiver 12 emits an unfocused beam with a focal zone covering the area of the prostate. Transmitter/receiver 12 scans a limit angle sector for angular coverage of the prostate. More preferably, multiple measurements are performed at different times, for example at periodic intervals of several months, in order to be able to detect changes and to decrease artifacts.

The power Doppler is the sum of all intensities of every component of the Doppler velocities. Since only the relative velocity is measured, obtaining the sum is important in order to detect any changes.

The envelope, the phase, frequency and the time of the back-scattered signal is preferably determined in DSP 16, with algorithms 22. The delay of the scattering signal can optionally be calculated with the internal modulation of the pulse.

A decaying exponential curve having the form y=exp(−Dt) is fitted to the envelope of DSP 16, where D is the attenuation factor for the curve and t is time. This part is preferably calibrated after initial measurements, as this calculation is an empirical calibration. The total back-scattered energy from the back signal is preferably measured. Also, the intensity and frequency and phase of the backscattered energy are preferably determined as function of time. The density profile of the prostate in one direction (the direction of the beam) is then preferably built. A density scale is then preferably generated, based on the attenuation factor and the total back-scattered energy as function of time and phase coherency. In addition, the Doppler profile is optionally calculated as function of distance in the beam direction.

In addition to the above data, an envelope signal is preferably determined as a function of time, frequency, and direction, by fitting a decaying exponential curve to the envelope, having the form y=exp(−Dt), where D is the attenuation factor for the curve and t is time. In the frequency domain, the Doppler data are calculated as function of the estimated prostate space. The space which is defined by angle and range is then compared to the ultrasound pen head. The dimensions of this space in the range and angle coordinates are stored in a matrix that contains the measured density and power Doppler for each point in the space. This matrix is compared to the previously stored matrix, used for the change detection algorithm. The receiving ultrasonic echo information associated with transmitted ultrasonic energy depends on the frequencies spectrum. The transmitted ultrasound pulse contains a spectrum of frequencies. The normal scattering coefficients of vessel boundaries and surrounding tissue are often on the order of 20 times larger than for blood, and a high pass filter is inserted in order to eliminate the DC component or by comparing the ratio in the different frequencies.

Algorithms 22 preferably determine a composite value for each divided space domain from the received ultrasonic echo pulse. Composite parameters for each cell in the space is calculated based on the detection of Doppler shift in the different transmitted frequencies and the relative intensities, from the Doppler power density spectrum. Such a comparison is preferably performed by dividing the directional space into cubes, and mapping the average intensity and velocity in the scattering direction and scale due to the attenuation factor of the tissue width in each cube. These measurements are different according to the tissue densities, as the cube is actually a cube in the r and angle dimensions. Therefore measurements are performed from a plurality of different angles using correlation and cross correlation functions.

Mapping the averaged Doppler parameters in the prostate gland space can be an important parameter for the early detection of cancer development in the gland. Since cancer tissue needs blood supply at the earliest stage of development, blood flow is increased in the cancerous tissue.

According to a preferred embodiment of the present invention, signals from two or more unique frequency bands or frequency modulation are processed separately to detect motion. The resulting Doppler values of each parameter, such as velocity, energy, and variance parameters, are averaged or otherwise combined. One of the average value, the Doppler values at each frequency band and a null value are selected for further processing and display. The resulting Doppler images may have improved accuracy and precision, without a loss in frame rate, resolution or displayed area. Further, the parameter selection process can be used to further improve differentiation between blood flow and clutter.

The signals at different frequency modulation are preferably used to further improve detection of velocities beyond the conventional limit. Energies of one frequency at higher frequencies is exposed to more significant attenuation than energies of a lower frequency, contributing to inferior SNR and potentially poor velocity estimates in some cases. Energy estimates are used to identify average SNR in the defined space. Energy estimates are screened to determine application of an algorithm to detect velocities beyond the individual conventional maximum velocity limits for either of the frequencies.

According to another aspect of the present invention, a plurality of pulses is transmitted along a first scan line. The plurality comprises a flow sample count. A plurality of samples responsive to the plurality of pulses, respectively, is received. The plurality of samples are separated into first and second sets of receive signals responsive to respective first and second frequency where a number of the first and second receive signals for each depth is at least about twice the flow sample count for a single frequency. First and second sets of Doppler values of a same parameter responsive to the first and second sets of receive signals, respectively, are determined. The first and second sets of Doppler values are combined, such that the combination increases an accuracy of the combined Doppler values.

According to yet another preferred embodiment of the present invention, an ultrasound method for Doppler processing is provided. First and second Doppler velocity values at first and second frequencies, respectively, are obtained where the first frequency is different than the second frequency. First and second Doppler energy values at the first and second frequencies, respectively, are also obtained. The application of an algorithm to extend the conventional maximum velocity limit associated with at least one of the first and second Doppler velocity values is determined in response to at least one of the first and second Doppler energy values.

According to optional but preferred embodiments of the present invention, the device is combined with a PSA (prostate specific antigen) diagnostic test. This test is a blood or urine screening test, which is preferably also performed once a year. The combination of these two sets of results is preferred, in order to increase the accuracy and specificity of the tests.

According to another optional embodiment of the present invention, the results can be optionally presented on a display, for example to display the change. The results may optionally be displayed as a 3 dimensional movie of the blood flow or a picture that is a slice-cut in the Doppler and density space. Another option is to present only a numerical display of threshold level of the power Doppler and the density. This type of display is known in the art, but has not been used for the detection of prostate cancer. The display is optionally and preferably connected to the device according to the present invention.

### REFERENCES

Ref-1. Burns P N. Interpreting and analyzing the Doppler examination. In: Taylor K J W, Bums P N, Wells P N T, eds. Clinical applications of Doppler ultrasound. New York, N.Y.: Raven, 1995; 55-98.

Ref 2. Wells P N T. Basic principles and Doppler physics. In: Taylor K J W, Burns P N, Wells P N T, eds. Clinical applications of Doppler ultrasound. New York, N.Y.: Raven, 1995; 1-17.

Ref 3 Jorgen Arendt Jensen, Estimation of Blood Velocities Using Ultrasound

Ref 4: Brem S. Angiogenesis and cancer control: From concept to therapeutic trial. Bio.com, 2001.

Ref 5: Folkman J., What is the evidence that tumors are angiogenesis dependent? J. Natl Cancer Inst., 1990, 82:4.

Ref 6: Folkman J, Klagsbrun M.: Angiogenesis factors, Science 1987, 235; 442-447.

Ref 7: The angiogenetic therapy self help book for cancer patients. 3^{rd} ed. Angiogenesis foundation Inc. Cambridge Mass. 1999.

Ref 8: Egawa S. et al. Impact of life expectancy and tumor doubling time on the clinical significance of prostate cancer in Japan. Jpn J. Clin Oncol 1997, 27, 394.

What is claimed is:

1. A device for detection of prostate cancer in a subject, comprising:
(a) an insertion portion for being inserted into the rectum of the subject; (b) a transmitter for transmitting an ultrasound signal to the rectum;
(c) a receiver for receiving reflected signal from tissue of the subject; and
(d) an analysis portion for analyzing said reflected signal to detect prostate cancer.

2. The device of claim 1, wherein a plurality of measurements of said reflected signal are performed and compared to analyze said reflected signal.

3. The device of claim 1, wherein said transmitter transmits said ultrasound signal in a plurality of frequencies, such that said analysis portion averages said plurality of frequencies for detection of prostate cancer.

4. The device of claim 3, wherein a plurality of energy values is obtained for analyzing said plurality of frequencies.

5. The device of claim 4, wherein a signal from said plurality of frequencies is analyzed to detect at least one of altered density and altered velocity.

6. The device of claim 1, further comprising a time varying filter for receiving a plurality of received signals and for selectively passing a portion of said received signals.

7. The device of claim 1, wherein the subject is a patient and the subject operates the device.

8. The device of claim 7, further comprising:
(e) a transmitter for transmitting data to a medical center.

9. The device of claim 1, further comprising a base for grasping said insertion portion, and wherein said insertion portion is attached to said base at a fixed angle, such that a location of said transmitter and said receiver is substantially fixed relative to said rectum.

10. The device of claim 9, wherein said fixed angle is alterable for each subject.

11. The device of claim 1, further comprising a display for displaying analyzed reflected signals, and wherein a plurality of ultrasound signals are transmitted and analyzed, such that a change in said analyzed reflected signals is displayed on said display.

12. A method for detecting prostate cancer in a subject with a device, comprising:
inserting an insertable portion of the device into the rectum of the subject;
transmitting ultrasound waves to the rectum;
receiving reflected signal from tissue of the subject; and
analyzing said reflected signal to detect prostate cancer.

13. The method of claim 12, wherein a plurality of transmissions of ultrasound waves is performed, at least two transmissions being separated by a period of time, and wherein said reflected signal is analyzed for said plurality of transmissions to detect at least one change in the subject, said at least one change being indicative of prostate cancer.

14. The method of claim 13, wherein said ultrasound waves are power Doppler ultrasound waves, and wherein said reflected signal is analyzed to detect said at least one change.

15. The method of claim 14, wherein said reflected signal is analyzed to further detect at least one change in blood flow and also a change in a density of the tissue of the subject, and wherein said at least one change in blood flow and said change in said density are correlated to detect prostate cancer.

16. The method of claim 15, wherein said power Doppler ultrasound waves are transmitted at a plurality of frequencies, and analyzing said reflected signal provides a plurality of energy values for detecting at least one of altered density of said tissue and altered blood flow velocity.

17. The method of claim 16, wherein analyzing said reflected signal further comprises selecting at least one suitable frequency for obtaining said energy value.

18. The method of claim 14, wherein analyzing said reflected signal further comprises processing said reflected signal before analysis, said processing comprising filtering said reflected signal with a FFT (fast Fourier transform) process.

19. The method of claim 18, wherein said processing further comprises filtering with at least one of a band pass filter and a low band filter.

20. The method of claim 19, wherein analyzing said reflected signal further comprises detecting a plurality of peaks of said reflected signal for calculating a power Doppler of said reflected signal.

21. The method of claim 12, wherein said ultrasound waves are analyzed at least according to Doppler ultrasound, to detect at least a change in a density of said tissue.
